(19) United States
(12) Patent Application Publication
(10) Pub. No.: US 2023/0135626 A1
(43) Pub. Date: May 4, 2023

(54) PHOTOCATALYST FILTER MODULE

(71) Applicant: WE.RB CO., LTD., Gimpo-si (KR)

(72) Inventors: Young Un BANG, Paju-si (KR); Kun Woo KIM, Goyang-si (KR)

(21) Appl. No.: 17/634,227

(22) PCT Filed: Jul. 1, 2021
(86) PCT No.: PCT/KR2021/008329

§ 371 Date: Feb. 9, 2022

(30) Foreign Application Priority Data
Mar. 12, 2021 (KR) 10-2021-0032775

(51) Int. Cl. A61L 9/20

(52) U.S. Cl. CPC A61L 9/205; A61L 2209/14; A61L 2209/12

(57) ABSTRACT

Disclosed is a photocatalyst filter module including: a housing having an inner space opened in a back-and-forth direction so that fluid passes therethrough; a partition for forming a plurality of photocatalyst accommodation spaces by partitioning the inner space; a photocatalytic ball accommodated in the photocatalyst accommodation space; a mesh-shaped cover unit attached to the front and the rear of the housing to prevent the photocatalytic ball from being separated; and a light source unit provided adjacent to the rear of the housing.

### TECHNICAL FIELD

The present invention relates to a photocatalyst filter module, and more specifically, to a photocatalyst filter module having a structure capable of allowing contaminated air to pass therethrough, inducing a photocatalytic reaction uniformly in all directions of a plurality of photocatalytic balls accommodated therein, and rapidly causing the photocatalytic reaction.

### BACKGROUND ART

Energy photocatalytic technique is to decompose various harmful substances and bacteria into harmless water and carbon dioxide and remove bad smell by causing an oxidation-reduction reaction using ultraviolet (UV) rays of the sun or a fluorescent lamp as a source of energy like photosynthesis which purifies a forest by generating oxygen using chlorophyl as a catalyst when receiving light.

FIG. 1 illustrates a conventional filter structure of an air purifier using photocatalytic technique.

As illustrated in FIG. 1, contaminated air (A1) flowing forward is introduced to the front of a pre-filter 1 due to the flow of air generated by a fan 5. Dust of an intermediate size is removed while the contaminated air (A1) passes through the pre-filter 1, and bad smell, fine dust a size of 0.3 micrometer and indoor molds are removed while the contaminated air passes through a carbon filter 2 and a HEPA filter 3. Moreover, harmful substances and pathogenic bacteria remaining in the contaminated air are decomposed into water and carbon dioxide, which are harmless to the human body, due to the photocatalytic reaction of a photocatalytic filter 4, and finally, purified air (A2) is discharged to the outside.

Meanwhile, as illustrated in FIG. 2, the conventional photocatalyst applied to the photocatalyst filter has a form of being thinly coated on a compressed ceramic substrate (See FIG. 2(a)) having pores or a silicon substrate (See FIG. 2(b)) having pores.

In such a porous structure, because UV light irradiated to the photocatalyst filter is difficult to pass through the pores of the photocatalytic filter, the decomposition rate of harmful substances in contaminated air passing through the photocatalyst filter is low since the UV light is not entirely irradiated to the photocatalyst filter.

Furthermore, due to fine pores, since the flow rate of air passing through the photocatalytic filter is low, it takes long time to sterilize.

Additionally, since the photocatalytic reaction is intensively generated on the side facing the UV light source, the photocatalytic filter must be frequently replaced due to the durability problem that some of the photocatalytic filter is crumbled.

In addition, the conventional photocatalyst has another disadvantage in that it requires lots of replacement costs since the unit price of the UV light source is high and the lifespan thereof is limited.

### DISCLOSURE

### Technical Problem

Accordingly, the present invention has been made in an effort to solve the above-mentioned problems occurring in the prior arts, and it is an object of the present invention to provide a photocatalyst filter module having a structure capable of allowing contaminated air to pass therethrough, inducing a photocatalytic reaction uniformly in all directions of a plurality of photocatalytic balls accommodated therein, and rapidly causing the photocatalytic reaction.

It is another object of the present invention to provide a photocatalyst filter module having excellent sterilization performance and low costs by using a UV light-A light source for causing a photocatalytic reaction.

The technical problem to be solved by the present invention is not limited to the technical problem as mentioned above, and another technical problem, which is not mentioned, could be clearly understood by those having ordinary skill in the art to which the present invention pertains based on the description below.

### Technical Solution

To achieve the above objects, the present invention provides a photocatalyst filter module including: a housing having an inner space opened in a back-and-forth direction so that fluid passes therethrough; a partition for forming a plurality of photocatalyst accommodation spaces by partitioning the inner space; a photocatalytic ball accommodated in the photocatalyst accommodation space; a mesh-shaped cover unit attached to the front and the rear of the housing to prevent the photocatalytic ball from being separated; and a light source unit provided adjacent to the rear of the housing.

The photocatalytic ball has a diameter of 10 mm or less.

The maximum width, the maximum height, and the maximum depth of the photocatalyst accommodation space are 1.2 times to three times the diameter of the photocatalytic ball.

The sum of the volumes of one or more photocatalytic balls accommodated in the photocatalyst accommodation space ranges from ½ to ¾ of the volume of each single photocatalyst accommodation space.

The cover unit includes: a first cover unit attached to the front of the housing; and a second cover unit attached to the rear of the housing.

The light source unit includes: a frame; and one or more light emitting units mounted in the frame, and the light emitting unit irradiates light toward the photocatalytic ball accommodated in the photocatalyst accommodation space.

An interval between the first cover unit of the housing and the light emitting unit is 30 mm or less.

The maximum depth of the photocatalyst accommodation space is 15 mm or less.

The light emitting unit further includes: an optical fiber, the light emitting unit irradiates light into the optical fiber, and the optical fiber is a side-emitting optical fiber.

The light emitting unit is a laser UV light source.

The frame has at least one window which is opened such that fluid pass through the window, and the optical fiber extends along the edge of the window.

The optical fiber is rolled in a shape of a pig tail.

The photocatalyst filter module further includes: a mesh-shaped protective cover unit disposed in the front of the first cover unit to reflect the light passing through the photocatalyst accommodation space so that the light irradiated from the light source unit and passing through the photocatalyst accommodation space heads the photocatalyst accommodation space again.

The protective cover unit is made of an aluminum material.

The protective cover unit comprises a plurality of first mesh ribs and a plurality of second mesh ribs which intersect with each other so as to form a mesh shape, and the plurality of first mesh ribs and the plurality of second mesh ribs are inclined from the partition in the width direction.

The plurality of first mesh ribs and the plurality of second mesh ribs are formed such that a surface opposed to the direction facing the light source unit is more convex than a surface facing the light source unit.

The plurality of first mesh ribs and the plurality of second mesh ribs are formed such that the surface facing the light source unit is flat.

The plurality of first mesh ribs and the plurality of second mesh ribs are formed such that the surface facing the light source unit is concave.

The protective cover units are formed in a pair. A pair of the protective cover units are stacked in the back-and-forth direction, and the first mesh ribs of one protective cover unit and the first mesh ribs of the other protective cover unit are arranged to be crossed with each other.

### Advantageous Effects

According to an embodiment of the present invention, the photocatalyst is formed in a ball shape having no pores therein, thereby maintaining a rapid flow rate of air passing through the photocatalyst accommodation space and allowing a sufficient level of photocatalytic reaction required for sterilization.

Moreover, the photocatalyst is formed in the form of a lump-shaped ball, thereby having excellent durability since being less crumbled.

Furthermore, according to an embodiment of the present invention, the manufacturing cost of the photocatalyst filter module can be lowered by using a UV light-A light source which emits only a relatively narrow wavelength of light required for a photocatalytic reaction as a light source for causing a photocatalytic reaction.

In addition, according to an embodiment of the present invention, the photocatalyst accommodation space has the maximum width, the maximum height, and the maximum depth of 1.2 times or more times the diameter of the photocatalytic ball, thereby enhancing sterilization effect since easily forming a vortex in the photocatalyst accommodation space.

Additionally, according to an embodiment of the present invention, the total volume of at least one photocatalytic ball accommodated in the single photocatalyst accommodation space is ½ to ¾ of the volume of the single photocatalyst accommodation space, so that the air flow rate in the photocatalyst accommodation space can be rapidly maintained.

Moreover, according to an embodiment of the present invention, since the interval between the first cover unit and the light emitting unit is 30 mm or less, the photocatalytic reaction can be evenly generated in the plurality of photocatalytic balls.

Furthermore, according to an embodiment of the present invention, the protective cover unit reflects the light irradiated from the light source unit and passing through the photocatalyst accommodation space toward the photocatalyst accommodation space, so that the photocatalytic reaction can be evenly generated in all directions of the plurality of photocatalytic balls.

In addition, according to an embodiment of the present invention, since the protective cover unit guides the path of the contaminated air to be inclined from the back-and-forth direction, it can promote formation of a vortex in the plurality of photocatalyst accommodation spaces.

Additionally, according to an embodiment of the present invention, since the plurality of first mesh ribs and the plurality of second mesh ribs are formed such that a surface opposed to the direction facing the light source unit is more convex than a surface facing the light source unit, resistance against the air introduced into the front of the protective cover unit can be minimized, and a probability that the light reflected from the protective cover unit heads the photocatalyst accommodation space.

In addition, according to an embodiment of the present invention, the photocatalyst filter module can generate a photocatalytic reaction in a plurality of photocatalyst accommodation spaces with a small number of light emitting units since including the optical fiber for evenly transferring light irradiated from the light emitting unit to the plurality of photocatalyst accommodation spaces.

It should be understood that the present invention is not limited to the above effects, and includes all effects that are inferred from the features and configuration of the invention as set forth in the description or the claims.

### DESCRIPTION OF DRAWINGS

FIG. 1 illustrates a conventional filter structure of an air purifier using a photocatalytic technique.

FIG. 2 is an enlarged view of a structure of a conventional photocatalyst.

FIG. 3 is a block diagram of a photocatalyst filter module according to an embodiment of the present invention.

FIG. 4 is a view illustrating a photocatalyst accommodation space formed in a housing according to an embodiment of the present invention.

FIG. 5 is a view for explaining a structural relationship between the photocatalyst accommodation space and a photocatalytic ball according to an embodiment of the present invention.

FIG. 6 is a view illustrating the diffusion level that the light irradiated from a light source unit is diffused to a plurality of photocatalyst accommodation spaces according to an interval between a first cover unit and the light source unit.

FIG. 7 is a view illustrating a protective cover unit according to an embodiment of the present invention.

FIG. 8 is a view illustrating an influence of the protective cover unit on light diffusion in the plurality of photocatalyst accommodation spaces.

FIG. 9 is an antero-posterior cross-sectional view of the protective cover unit.

FIG. 10 is a view illustrating an optical fiber of the light source unit according to an embodiment of the present invention.

### BEST MODE

Hereinafter, the present invention will be described in detail with reference to the accompanying drawings. However, embodiments of the present invention may be implemented in several different forms and are not limited to the embodiments described herein. In addition, parts irrelevant to description are omitted in the drawings in order to clearly explain embodiments of the present invention. Similar parts are denoted by similar reference numerals throughout this specification.

Throughout this specification, when a part is referred to as being “connected” to another part, this includes “direct connection” and “indirect connection” via an intervening part. Also, when a certain part “includes” a certain component, other components are not excluded unless explicitly described otherwise, and other components may in fact be included.

The terminology used herein is for the purpose of describing particular embodiments only and is not intended to be limiting. As used herein, the singular forms are intended to include the plural forms as well, unless the context clearly indicates otherwise. It will be further understood that the terms “comprise”, “include”, “have”, etc. when used in this specification, specify the presence of stated features, integers, steps, operations, elements, components, and/or combinations of them but do not preclude the presence or addition of one or more other features, integers, steps, operations, elements, components, and/or combinations thereof.

Hereinafter, embodiments of the present invention will be described in detail with reference to the accompanying drawings.

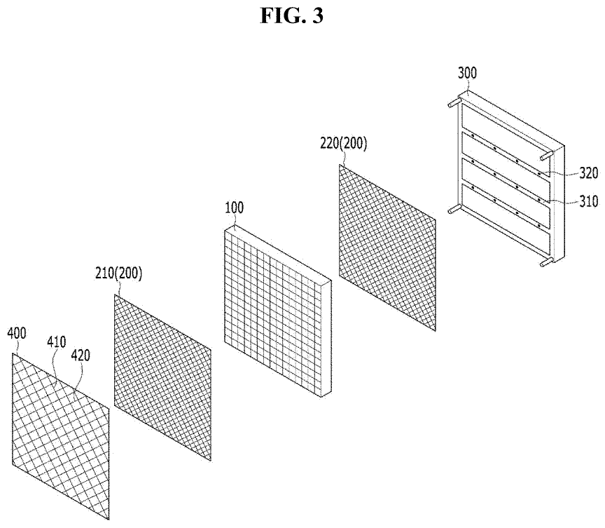

FIG. 3 is a block diagram of a photocatalyst filter module according to an embodiment of the present invention.

As illustrated in FIG. 3, the photocatalytic filter module may include a housing 100, a cover unit 200, a light source unit 300, and a protective cover unit 400.

The housing 100 is a substrate for receiving photocatalytic balls (B), and can accommodate photocatalytic balls (B) in an inner space. The inner space of the housing 100 is opened in the back-and-forth direction to allow fluid to pass therethrough. For example, contaminated air (A1) can be introduced to the front of the inner space and pass through the back of the inner space, and in this process, is discharged out after being converted into purified air (A2) by hydroxyl radicals generated through the photocatalytic reaction.

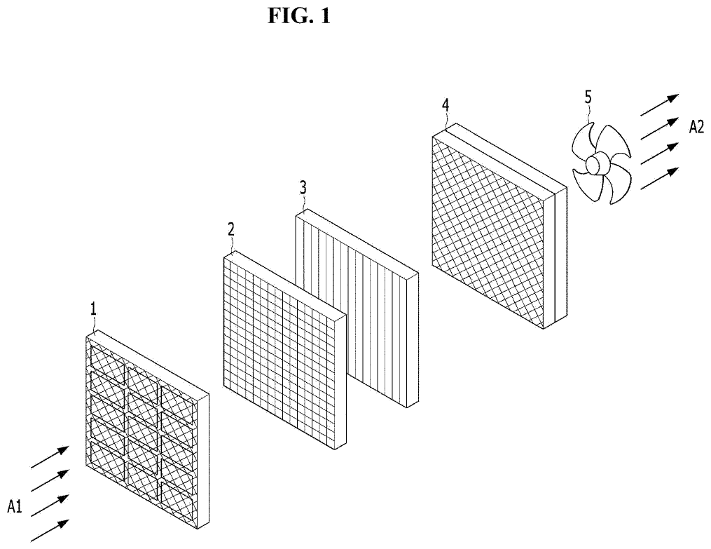

The flow of air can be formed by a separate device, such as a fan 5 illustrated in FIG. 1.

Moreover, the inner space of the housing 100 can be partitioned into a plurality of photocatalyst accommodation spaces. In addition, a plurality of photocatalytic balls (B) can be accommodated in each photocatalyst accommodation space.

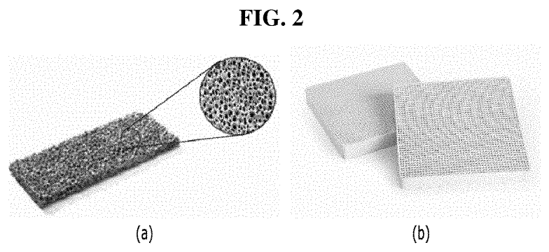

In particular, according to an embodiment of the present invention, unlike the photocatalyst of FIG. 2 which is coated on a porous substrate, the photocatalytic ball (B) which is processed by agglomerating photocatalysts into a lump can be formed in the form of a ball having no internal pores.

Compared with the photocatalyst of FIG. 2 having a porous structure, the photocatalytic ball (B) allows a rapid flow rate of the contaminated air passing through the photocatalyst accommodation space and can obtain a sufficient level of photocatalytic reaction required for sterilization.

Furthermore, the photocatalyst of FIG. 2 is formed to be thinly coated on a porous substrate, but the photocatalytic ball (B) according to an embodiment of the present invention has excellent durability because the photocatalyst is formed in a lump shape and is less crumbled by the photocatalytic reaction.

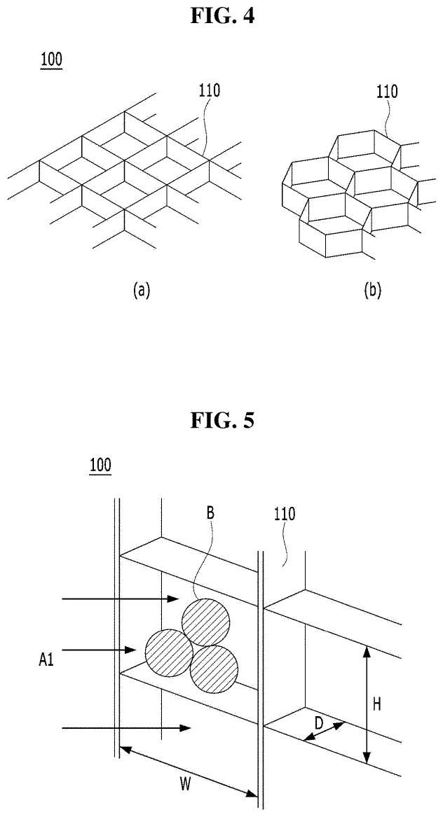

The specific structure of the photocatalyst accommodation space in which the photocatalytic balls (B) are accommodated will be described in detail with reference to FIG. 4.

The cover unit 200, which is a member for preventing the photocatalytic balls (B) from being separated from the photocatalyst accommodation space, can be attached to the front and the rear of the housing 100. For example, the cover unit 200 may include a first cover unit (210) attached to the front of the housing 100, and a second cover unit (220) attached to the rear of the housing 100.

On the other hand, the single cover unit 200 having a “⊏” shape is attached to the housing 100 to simultaneously cover the front and the rear of the housing 100.

The light source unit 300 is provided at the rear of the housing 100 to irradiate light toward the housing 100.

For example, the light source unit 300 may include a frame 310 and a light emitting unit 320. The light emitting unit 320 is mounted on the frame 310 to irradiate light toward the photocatalytic balls (B) in the photocatalyst accommodation space. For example, the light emitting unit 320 may be a light source for irradiating ultraviolet rays.

For example, the light emitting unit 320 may be a UV light-A light source. Unlike a general UV light source emitting light in a wide wavelength range, the UV light-A light source has an advantage of lowering manufacturing costs of the photocatalyst filter module since emitting light having a relatively narrow wavelength required for a photocatalytic reaction and reducing a unit cost. As an example, the UV light-A light source may be selected from light sources having a lifetime in the range of about two to five million hours.

In addition, one or more windows through which fluid can pass can be formed in the frame 310.

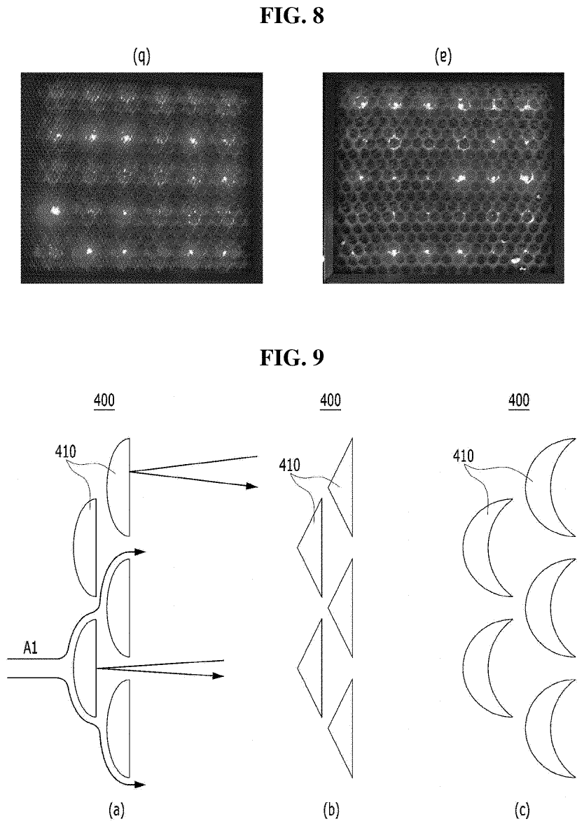

The protective cover unit 400 can be provided in front of the housing 100. The protective cover unit 400 protects the rear structure from an external impact or foreign matter and simultaneously reflects light which has passed through the photocatalyst accommodation space by being irradiated from the light source unit 300. The features of the protective cover unit 400 will be described in detail with reference to FIGS. 8 to 9.

FIG. 4 is a view illustrating a photocatalyst accommodation space formed in a housing 100 according to an embodiment of the present invention.

As illustrated in FIG. 4(a), the inner space of the housing 100 can be partitioned into a plurality of photocatalyst accommodation spaces.

As an example, the inner space of the housing 100 can be partitioned by a partition 110. That is, the partition 110 divides the inner space of the housing 100 to form a plurality of photocatalyst accommodation spaces.

According to an embodiment of the present invention, the partition 110 may include a plurality of first partitions having a shape extending in a vertical direction and a plurality of second partitions having a shape extending in the lateral direction. The plurality of first partitions and the plurality of second partitions may cross each other, and a plurality of photocatalyst accommodation spaces may be formed.

Additionally, photocatalytic balls (B) can be accommodated in each photocatalyst accommodation space. According to an embodiment of the present invention, one or more photocatalytic balls (B) can be accommodated in the single photocatalyst accommodation space.

Meanwhile, as illustrated in FIG. 4(b), the plurality of photocatalyst accommodation spaces may be formed in a honeycomb shape. Although not shown in FIG. 4, the plurality of photocatalyst accommodation spaces may be formed in a circular shape.

FIG. 5 is a view for explaining a structural relationship between the photocatalyst accommodation space and the photocatalytic ball according to an embodiment of the present invention.

As illustrated in FIG. 5, the contaminated air (A1) can enter the front of the photocatalyst accommodation space and pass through the photocatalyst accommodation space.

In this instance, in order to sufficiently sterilize the contaminated air (A1), the contaminated air (A1) must stay in the vicinity of the photocatalyst accommodation space for a predetermined period of time or longer. In other words, the photocatalyst accommodation space must be designed to have a structure in which the air entering the front side can stay for a predetermined period of time till passing through the photocatalyst accommodation space in which the photocatalytic balls (B) are accommodated.

To achieve the above, the photocatalyst accommodation space may have a maximum width (W) of 1.2 times to three times the diameter of a photocatalytic ball (B). Moreover, the photocatalyst accommodation space may have a maximum height (H) of 1.2 times to three times the diameter of a photocatalytic ball (B). Furthermore, the photocatalyst accommodation space may have a maximum depth (D) of 1.2 times to three times the diameter of a photocatalytic ball (B). Such a condition is to induce the formation of vortex in the photocatalyst accommodation space by accommodating the plurality of photocatalytic balls (B) in the photocatalyst accommodation space. When the vortex is formed, the contaminated air (A1) can be effectively sterilized since time to stay in the vicinity of the photocatalytic balls (B) is increased.

Additionally, the entire volume of the photocatalytic ball (B) accommodated in the single photocatalyst accommodation space may be ½ to ¾ of the volume of the single photocatalyst accommodation space. Since the photocatalytic ball (B) occupies a physical space, if too many photocatalytic balls (B) are accommodated in the single photocatalyst accommodation space, the flow of the fluid is excessively suppressed.

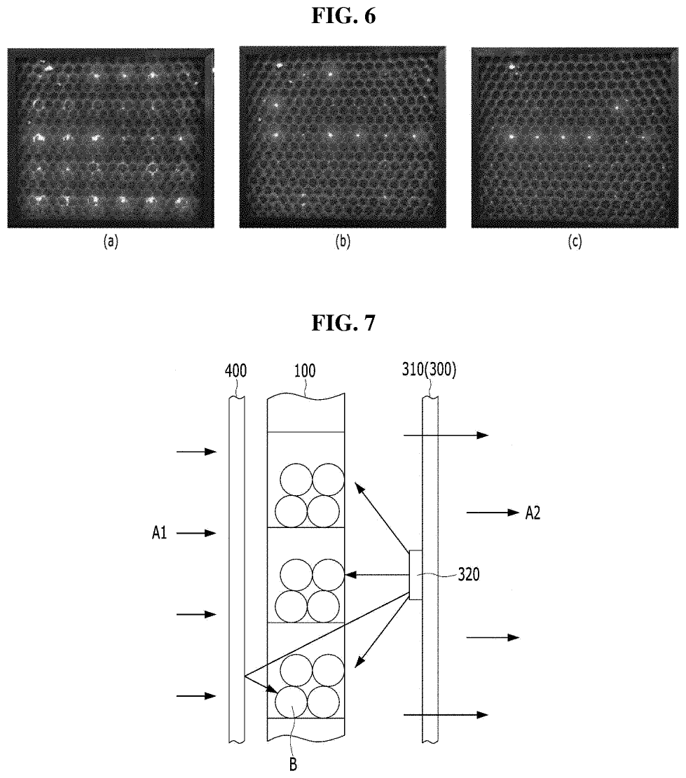

Meanwhile, FIG. 6 is a view for explaining a diffusion level of light emitted from the light source unit 300 to the plurality of photocatalyst accommodation spaces according to an interval between the first cover unit 210 and the light source unit 300.

In detail, FIG. 6(a) is a front view of the photocatalyst filter module in a state where an interval between the light emitting unit 320 mounted on the light source unit 300 and the first cover unit 210 is 20 mm. Moreover, FIG. 6(b) is a front view of the photocatalyst filter module in a state where an interval between the light emitting unit 320 mounted on the light source unit 300 and the first cover unit 210 is 30 mm. Furthermore, FIG. 6(c) is a front view of the photocatalyst filter module in a state where an interval between the light emitting unit 320 mounted on the light source unit 300 and the first cover unit 210 is 40 mm.

In the embodiment of FIG. 6(c), the light irradiated from the light source unit 300 is diffused into a portion of the plurality of photocatalyst accommodation spaces. On the other hand, in the embodiment of FIGS. 6(a) and 6(b), the light irradiated from the light source unit 300 is uniformly diffused to the plurality of photocatalyst accommodation spaces, differently from the embodiment of FIG. 6(c).

The probability that the contaminated air (A1) is sterilized while passing through the photocatalyst accommodation space can be increased as the photocatalytic reaction occurs evenly in the plurality of photocatalytic balls (B). Therefore, the interval between the first cover unit 210 and the light emitting unit 320 is preferably 30 mm or less.

Meanwhile, in order to maintain the interval between the first cover unit 210 and the light-emitting unit 320 at 30 mm or less and to satisfy the condition of FIG. 5, the diameter of the photocatalytic ball (B) can be 10 mm or less. In addition, the maximum depth of the photocatalyst accommodation space can be 15 mm or less.

The contaminated air (A1) passing through the photocatalyst accommodation space can be sufficiently sterilized while maintaining a proper flow rate when the structural conditions of the photocatalyst accommodation space and the photocatalytic ball (B) set forth in FIGS. 5 and 6 are satisfied.

According to an experimental result, when air in a predetermined space was sterilized for 30 minutes in the state in which the condition of FIGS. 5 and 6 was satisfied, viruses in the air were not detected. On the other hand, when the above condition was not satisfied, a number of viruses in the air were detected under the same experimental conditions. That is, it has been experimentally confirmed that the sterilization effect was rapidly increased when the condition of FIGS. 5 and 6 was satisfied.

FIG. 7 is a view illustrating the protective cover unit 400 according to an embodiment of the present invention.

As illustrated in FIG. 7, the protective cover 400 may be provided in front of the housing 100. Moreover, the protective cover unit 400 can be formed in a mesh shape so as to allow fluid to pass therethrough. Furthermore, the protective cover unit 400 can protect the housing 100 and the first cover unit 210 from an external impact or foreign matter.

Additionally, the protective cover unit 400 can reflect light which was irradiated from the light source unit 300 and passed through the photocatalyst accommodation space. In this instance, the light reflected by the protective cover unit 400 can head the photocatalyst accommodation space. Therefore, the light irradiated from the light source unit 300 can be more evenly diffused into the plurality of photocatalyst accommodation spaces. Therefore, the photocatalytic reaction can be evenly generated in all directions of the plurality of photocatalytic balls (B).

In addition, the protective cover unit 400 can be made of a material which is able to reflect the light emitted from the light source unit 300, namely, ultraviolet rays, and have durability against ultraviolet rays. For example, the protective cover unit 400 can be made of an aluminum material.

Referring to FIG. 3, the protective cover unit 400 may include a plurality of first mesh ribs 410 and a plurality of second mesh ribs 420 extending in different directions. In this instance, since the first mesh rib 410 and the second mesh rib 420 intersect with each other, the protective cover unit 400 can have a mesh shape.

Moreover, the plurality of first mesh ribs 410 and the plurality of second mesh ribs 420 can be inclined from the partition 110 in the width direction. For example, when the first mesh rib 410 slantly extends to the left from the vertical direction, the second mesh rib 420 slantly extends to the right from the vertical direction. That is, the protective cover unit 400 can be formed in a net shape which is inclined diagonally.

According to the present invention, the protective cover unit 400 can disperse the contaminated air (A1) introduced to the front of the photocatalyst filter module in various directions. That is, the protective cover unit 400 can guide the contaminated air (A1) to be dispersed into the plurality of photocatalyst accommodation spaces.

In addition, since the protective cover unit 400 guides the path of the contaminated air (A1) to be inclined from the back-and-forth direction, the formation of vortex in the plurality of photocatalyst accommodation spaces can be promoted.

FIG. 8 is a view for explaining the influence of the protective cover unit 400 according to an embodiment of the present invention on light diffusion in the plurality of photocatalyst accommodation spaces.

In detail, FIG. 8(a) is a front view of the photocatalyst filter module in a state in which the protective cover unit 400 is not provided. In addition, FIG. 8(b) is a front view of the photocatalyst filter module in a state in which the protective cover unit 400 is provided in front of the first cover unit 210.

Comparing the embodiment of FIG. 8(a) and the embodiment of FIG. 8(b) with each other, it is confirmed that the light inside the plurality of photocatalyst accommodation spaces of the embodiment of FIG. 8(b) is diffused more uniformly than the light inside the plurality of photocatalyst accommodation spaces of the embodiment of FIG. 8(a).

FIG. 9 is an antero-posterior cross-sectional view of the protective cover unit 400 according to an embodiment of the present invention.

FIG. 9 shows a cross-section of the first mesh frame 410 for convenience of description, and the second mesh rib 420 may also have the same cross-sectional shape as the first mesh frame 410 shown in FIG. 9.

As illustrated in FIG. 9(a), the light reaching the protective cover unit 400 after passing through the photocatalyst receiving space can be reflected and head the photocatalyst receiving space.

In this instance, the plurality of first mesh ribs 410 and the plurality of second mesh ribs 420 may have a structure to increase the probability that the contaminated air (A1) introduced forward easily passes through the photocatalyst accommodation space and the light reflected by the protective cover unit 400 heads the photocatalyst accommodation space.

For instance, the plurality of first mesh ribs 410 and the plurality of second mesh ribs 420 can be formed such that a surface opposed to the direction facing the light source unit 300 is more convex than a surface facing the light source unit 300.

Therefore, the air introduced to the front of the protective cover unit 400 is guided to face the photocatalyst accommodation space along the convex surface of the first mesh rib 410 or the second mesh rib 420, so that the air resistance of the protective cover unit 400 can be minimized.

Since the convex surface increases the probability that the path of the contaminated air (A1) reaching the first mesh rib 410 or the second mesh rib 420 heads the rear, the formation of vortex in the plurality of photocatalyst accommodation spaces can be promoted.

Moreover, since the light passing through the photocatalyst accommodation space is radiated to the rear surface of the first mesh rib 410 or the second mesh rib 420 more concave than the front, the light reflected by the protective cover unit 400 can head the optical catalyst accommodation space with a higher probability.

As another example, as illustrated in FIG. 9(b), the plurality of first mesh ribs 410 and the plurality of second mesh ribs 420 may be formed such that the surface facing the light source unit 300 is flat.

As another example, as illustrated in FIG. 9(c), the plurality of first mesh ribs 410 and the plurality of second mesh ribs 420 may be formed such that the surface facing the light source unit 300 is concave.

Meanwhile, the protective cover unit 400 may be formed in a pair.

For example, a pair of protective cover units 400 can be stacked in the back-and-forth direction. For instance, the first mesh ribs 410 of one protective cover unit 400 and the first mesh ribs 410 of the other protective cover unit 400 can be arranged to be crossed with each other.

According to the present invention, the protective cover unit 400 allows contaminated air (A1) introduced from the front to easily pass and can reflect the light, which was irradiated from the light source unit 300 and passed through the photocatalyst accommodation space, to the photocatalyst accommodation space with a higher probability.

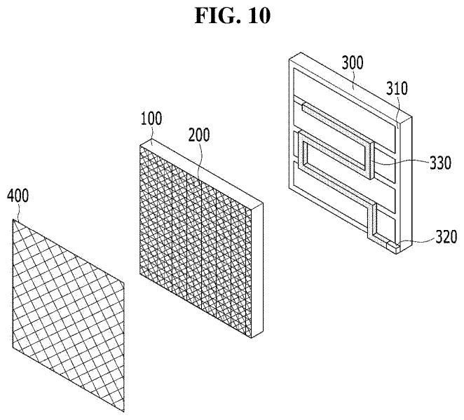

FIG. 10 illustrates an optical fiber 330 of the light source unit 300 according to an embodiment of the present invention.

As illustrated in FIG. 10, the light source unit 300 may include an optical fiber 330 connected to the light emitting unit 320.

The optical fiber 330 can be formed in the shape of a tube and can be elongated along the frame 310. For example, the optical fiber 330 can be formed in a shape extending along the edge of the window formed in the frame 310.

In addition, the light emitting unit 320 can irradiate light to the interior of the optical fiber 330. The light incident into the optical fiber 330 can be moved along an optical path inside the optical fiber 330.

In this instance, the light moving inside the optical fiber 330 can reach the inner side surface of the optical fiber 330 and some of the light reaching the inner side surface can be emitted to the outside. Furthermore, the light emitted to the outside can be irradiated toward the photocatalyst accommodation space. For example, the optical fiber 330 may be a side-emitting optical fiber 330.

The optical fiber 300 can relatively evenly deliver the light emitted from the light-emitting unit 320 to the plurality of photocatalyst accommodation spaces even when the number of the light-emitting units 320 is small.

That is, since a photocatalytic reaction can be generated in the plurality of photocatalytic balls (B) by using a small number of light emitting units 320, the power consumption required for driving the photocatalyst filter module can be lowered. Furthermore, since the optical fiber 300 can be formed to be thin, the overall thickness of the photocatalytic filter module can get thinner. For example, the light emitting unit 320 may be a laser infrared light source.

Additionally, although not shown in FIG. 10, at least a portion of the optical fiber 330 may be formed in a shape of a pig tail. This is because the ratio of the light emitted to the outside through the side surface of the optical fiber 330 is different according to the bending degree of the optical fiber 330.

In other words, when the optical fiber 330 is formed to be rolled like a pig tail, the bending change rate of the optical fiber 330 gets lower, so that light emission at the side of the optical fiber 330 can also be relatively uniform.

The above description of the present disclosure is just for illustration, and a person skilled in the art will understand that the present disclosure can be easily modified in different ways without changing essential techniques or features of the present disclosure. Therefore, the above embodiments should be understood as being descriptive, not limitative. For example, any component described as having an integrated form may be implemented in a distributed form, and any component described as having a distributed form may also be implemented in an integrated form.

The scope of the present disclosure is defined by the appended claims, rather than the above description, and ail changes or modifications derived from the meaning, scope and equivalents of the appended claims should be interpreted as falling within the scope of the present disclosure.

### MODE FOR INVENTION

The configuration for the practice of the invention has been described in conjunction with the best form.

### INDUSTRIAL APPLICABILITY

According to an embodiment of the present invention, photocatalyst filter module having a structure capable of allowing contaminated air to pass therethrough, inducing a photocatalytic reaction uniformly in all directions of a plurality of photocatalytic balls accommodated therein, and rapidly causing the photocatalytic reaction. So, the photocatalyst filter module according to an embodiment of the present invention can be used by being mounted in an air purifier in various industrial sites requiring sterilization and purification of air.

1. A photocatalyst filter module comprising:
a housing having an inner space opened in a back-and-forth direction so that fluid passes therethrough;
a partition for forming a plurality of photocatalyst accommodation spaces by partitioning the inner space;
a photocatalytic ball accommodated in the photocatalyst accommodation space;
a mesh-shaped cover unit attached to the front and the rear of the housing to prevent the photocatalytic ball from being separated; and
a light source unit provided adjacent to the rear of the housing.

2. The photocatalyst filter module according to claim 1, wherein the photocatalytic ball has a diameter of 10 mm or less.

3. The photocatalyst filter module according to claim 1, wherein the maximum width, the maximum height, and the maximum depth of the photocatalyst accommodation space are 1.2 times to three times the diameter of the photocatalytic ball.

4. The photocatalyst filter module according to claim 1, wherein the sum of the volumes of one or more photocatalytic balls accommodated in the photocatalyst accommodation space ranges from ½ to ¾ of the volume of each single photocatalyst accommodation space.

5. The photocatalyst filter module according to claim 1, wherein the cover unit comprises:
a first cover unit attached to the front of the housing; and
a second cover unit attached to the rear of the housing.

6. The photocatalyst filter module according to claim 5, wherein the light source unit comprises:
a frame; and
one or more light emitting units mounted in the frame, and
wherein the light emitting unit irradiates light toward the photocatalytic ball accommodated in the photocatalyst accommodation space.

7. The photocatalyst filter module according to claim 6, wherein an interval between the first cover unit of the housing and the light emitting unit is 30 mm or less.

8. The photocatalyst filter module according to claim 7, wherein the maximum depth of the photocatalyst accommodation space is 15 mm or less.

9. The photocatalyst filter module according to claim 6, wherein the light emitting unit further comprises:
an optical fiber,
wherein the light emitting unit irradiates light into the optical fiber, and
wherein the optical fiber is a side-emitting optical fiber.

10. The photocatalyst filter module according to claim 9, wherein the light emitting unit is a laser UV light source.

11. The photocatalyst filter module according to claim 9, wherein the frame has at least one window which is opened such that fluid pass through the window, and
wherein the optical fiber extends along the edge of the window.

12. The photocatalyst filter module according to claim 9, wherein the optical fiber is rolled in a shape of a pig tail.

13. The photocatalyst filter module according to claim 5, further comprising:
a mesh-shaped protective cover unit disposed in the front of the first cover unit to reflect the light passing through the photocatalyst accommodation space so that the light irradiated from the light source unit and passing through the photocatalyst accommodation space heads the photocatalyst accommodation space again.

14. The photocatalyst filter module according to claim 13, wherein the protective cover unit is made of an aluminum material.

15. The photocatalyst filter module according to claim 13, wherein the protective cover unit comprises a plurality of first mesh ribs and a plurality of second mesh ribs which intersect with each other so as to form a mesh shape, and wherein the plurality of first mesh ribs and the plurality of second mesh ribs are inclined from the partition in the width direction.

16. The photocatalyst filter module according to claim 15, wherein the plurality of first mesh ribs and the plurality of second mesh ribs are formed such that a surface opposed to the direction facing the light source unit is more convex than a surface facing the light source unit.

17. The photocatalyst filter module according to claim 16, wherein the plurality of first mesh ribs and the plurality of second mesh ribs are formed such that the surface facing the light source unit is flat.

18. The photocatalyst filter module according to claim 16, wherein the plurality of first mesh ribs and the plurality of second mesh ribs are formed such that the surface facing the light source unit is concave.

19. The photocatalyst filter module according to claim 15, wherein the protective cover units are formed in a pair, and
wherein a pair of the protective cover units are stacked in the back-and-forth direction, and the first mesh ribs of one protective cover unit and the first mesh ribs of the other protective cover unit are arranged to be crossed with each other.
